(19) United States
(12) Patent Application Publication
(10) Pub. No.: US 2004/0028624 A1
(43) Pub. Date: Feb. 12, 2004

(54) Chemically curing dental bleaching material

(75) Inventors: Alexander Bublewitz, Herborn (DE); Jens-Peter Reber, Herborn (DE); Matthias Suchan, Hachenburg (DE)

Correspondence Address: JACOBSON HOLMAN PLLC, 400 SEVENTH STREET N.W., SUITE 600, WASHINGTON, DC, 20004, US

(73) Assignee: Kettenbach GmbH & Co. Kg.

(21) Appl. No.: 10/426,022

(22) Filed: Apr. 30, 2003

Related U.S. Application Data
(63) Continuation of application No. 09/891,223, filed on Jun. 26, 2001, now abandoned.

(30) Foreign Application Priority Data
May 17, 2001 (EP) 01 112 127.4

(51) Int. Cl. A61K 7/20

(52) U.S. Cl. 424/53

(57) ABSTRACT

The present invention relates to a dental bleaching material which contains at least one bleaching substance and at least one support material capable of solidifying by a chemical reaction, and methods for the bleaching of vital or non-vital teeth.

The present invention relates to a dental bleaching material which contains at least one bleaching substance and at least one support material capable of solidifying by a chemical reaction, and methods for the bleaching of vital or non-vital teeth, the treatment of dental plaque, gingivitis and other oral and periodental diseases which respond to substances having a chemically oxidizing effect.

In recent years, methods for the bleaching of teeth have become wide-spread. These are methods of dentistry and cosmetics in which the teeth are brightened by the action of suitable chemical compounds.

One-component and two-component systems have been used. In the one-component systems, a hydrogen peroxide addition compound, such as carbamide peroxide, is embedded in an anhydrous matrix. This matrix consists of substances which are common in the galenics field, such as polyethylene glycols (PEG). The known bleaching materials are mostly provided as gels. After the application of the gel in the oral region, the water in the oral mucosa and in the saliva causes decomposition of the complex containing the hydrogen peroxide and results in the slow release of hydrogen peroxide, which acts as a bleaching agent.

In two-component systems (U.S. Pat. No. 5,928,628; U.S. Pat. No. 6,106,812; U.S. Pat. No. 6,110,446; PCT/US 99/22875), one component contains hydrogen peroxide which is in a gelled form in a matrix such as PEG. The second component contains highly dispersed silicic acid and alkaline earth carbonates. Prior to the application, the two components are mixed wherein the decomposition of H₂O₂ into aggressive free radicals having a strong bleaching effect is promoted, inter alia, by the presence of divalent ions, such as Ca²⁺.

PCT/US 99/06146 describes a dental bleaching composition which is capable of causing an increase in viscosity through an increase in temperature in order to prevent leaching out of the bleaching gel during the wearing time in the patient's mouth. However, this is not irreversible chemical curing.

The common methods can be carried out by a dentist. However, most frequent are the so-called “home bleaching methods” which can be performed by the patient at home under the control by a dentist. First, the dentist prepares a bar with the aid of a dental technician. Thus, an impression of the upper and lower jaws is made. The cured impression is removed from the patient's mouth, and plaster is cast into to prepare a positive impression. A stand-in material is mostly applied to the finished plaster cast in the region of the tooth surfaces to be brightened. Subsequently, a bar is prepared from plastic plates by a deep-drawing method, and fitted. The stand-in material is removed from the finished bar, and the cavities formed serve as a reservoir for a bleaching material to be introduced later. Now, the patient can take the bar home, independently fill it with a bleaching gel and perform a bleaching treatment on his own for some time by inserting it into his mouth.

With such a home bleaching method, the patient must go to the dentist twice, namely for preparing the impression and for receiving the bar. Subsequently, he can repeatedly perform the application of the bleaching agent on his own at home.

On the other hand, it is still relatively tedious for the user to be able to begin with the application of the bleaching agent only after the dentist has prepared an impression, the dental technician has processed it into a bar via a plaster cast, and the user has received the bar. In addition, after each application, the user must remove the tacky gel from the dental surface and clean the bar. Since the gel also penetrates into the spaces between the teeth, the expenditure for cleaning is correspondingly high, and it is not ensured for certain that the bleaching gel is completely removed. Another practical drawback is that the user, as we know from experience, tends to use too much gel when he introduces the gel into the bar, so that it will well out when the teeth are inserted into the bar. Thus, the aggressive bleaching substance comes into contact with the gum, spreads over the oral cavity and may be swallowed. Its action on, for example, exposed dental necks has the undesirable side effect that the sensitivity of the teeth increases. This phenomenon is frequently encountered for some days and, although reversible, adversely affects the user's well-being heavily. Even if the patient does not fill too much gel into the bar, the use of the described bar does not allow for an application in which the teeth rather than the adjacent gum are selectively contacted with the bleaching agent. Therefore, in the home bleaching method described, only relatively weak bleaching agents can be used for safety reasons, which involves the disadvantage that the user will have to apply to bleaching material more often.

Another drawback of the known bleaching materials is that the substances having a bleaching effect, such as carbamide peroxide, are relatively unstable because they are inactivated by the contact with water, e.g., atmospheric humidity.

Therefore, they must be provided in an anhydrous form, and after an air-tight package has been opened, they can be kept only for a limited period of time.

It has been the object of the invention to provide bleaching agents and methods for their application which avoid the drawbacks described. In particular, it would be desirable to provide materials and methods which can be applied by the user at home with a high specificity for the dental portions to be bleached and with high effectiveness in a gentle way. At the same time, the user is to spend as little time as possible at the dentist's before he can perform the first treatment.

The object of the invention is surprisingly achieved by a dental bleaching material which contains at least one bleaching substance and at least one support material, wherein the support material can be solidified by a chemical reaction.

As the bleaching substance, the bleaching material of the invention preferably contains hydrogen peroxide, carbamide peroxide, phthalimidoperoxohexanoic acid, alkali percarbonate, alkali perborate, alkali peroxodisulfate, peroxyacetic acid, alkali hypochlorite, and/or peroxides of organic acids or their salts. Further, addition compounds of hydrogen peroxide may be employed.

Preferred support materials include alginates, alginic acids, acrylates, methacrylates, acrylic acid, methacrylic acid, acrylamide, vinyl acetate, N-vinylpyrrolidones, methyl vinyl ether maleate, aziridines, vinyl ether, epoxides, polyols, polyamines, di- and polyisocyanates, cyanoacrylates, hydroxypolydialkylsiloxanes, alkenyl-substituted polyethers, polyethers and polysiloxanes containing SiH and Si-vinyl, polysulfides, glass ionomer filling materials, glass ionomer cements, zinc phosphate cements, carboxylate cements, silicate cements and silicophosphate cements.

The solidification of the support materials can be effected by usual cross-linking and/or polymerization methods, such as ionic cross-linking (e.g., alginate), felting by crystallization (e.g., plaster), free-radical polymerization (e.g., (meth)acrylate polymerization), polyaddition (e.g., polyurethanes), cationic polymerization (e.g., aziridines), anionic polymerization (e.g., epoxides), hydrosilylation and condensation reactions (e.g., polysulfides). After solidification by a chemical reaction, the support materials are preferably calcium alginate, polyacrylate, polymethacrylate, polyacrylic acid, polyvinylpyrrolidone, polyacrylamide, acrylic acid/acrylamide copolymers, polyurethanes, polyureas, polyethylene oxide/polypropylene oxide copolymers, poly(methyl vinyl ether maleate), polyamides, polyethylene glycols and polypropylene glycols, polysulfides, cross-linked polydialkylsiloxanes, and mixtures and copolymers thereof.

Preferably, the bleaching material according to the invention is provided as a multicomponent system containing two or more components. Particularly advantageous is a two-component system consisting of two components A and B.

Preferably, component A contains at least one bleaching substance, and component B contains said at least one support material.

For a two-component system, it is particularly advantageous if the bleaching substance from component A catalyzes the solidification of the support material from component B after mixing. This is the case, for example, when bleaching substances such as hydrogen peroxide or other peroxo compounds catalyze the free-radical polymerization of methacrylates or acrylates.

The bleaching materials according to the invention can additionally contain reinforcing and/or non-reinforcing fillers, reaction inhibitors, di- or trivalent salts, wetting agents, surfactants, emulsifiers, alcohols, water, solvents, buffer systems, colorants, fragrances, gelling agents and/or substances for increasing the viscosity (thickeners and paste formers). Generally, those additives can be used which the skilled person knows to be suitable for improving or adjusting the tolerance within the oral cavity, the quality (viscosity, dischargeability) or the optical appearance or taste impression.

Common additives also include glycols, glycerol, polyethylene glycols; propylene glycols, diethylene glycols, polypropylene glycols, fatty alcohol ethoxylates, polyvinylpyrrolidone, cross-linked polyvinylpyrrolidone, copolymers of N-vinylpyrrolidone and vinyl acetate, polyacrylic acid or its alkali or alkaline earth salts, carboxymethyl-, methyl-, hydroxyethyl-, hydroxypropylcellulose and polysaccharides, silica, kieselguhr, diatomaceous earth, colorants or colorant systems which undergo a color change upon changes of pH or redox potential.

The bleaching materials according to the invention and components of two- or multicomponent systems can be in the form of a gel, liquid, powder or paste.

For two-component systems, combinations are preferred in which components A and B are in the forms, respectively, of a gel and a gel, a liquid and a liquid, a powder and a powder, a paste and a paste, a gel and a liquid, a gel and a paste, a paste and a liquid, or a powder and a paste.

When the bleaching material according to the invention is used in a home bleaching method, its simple applicability is of importance. In this case, the user preferably has two components available for which he achieves a thorough mixing in a simple way with little expenditure of time and force, so that the bleaching material is as homogeneous as possible before being subsequently applied to the teeth.

The solidification of the bleaching material is effected after application thereof to the teeth to be bleached. After the solidification of the support material, the bleaching materials are in a plastic, non-elastic solid or elastic solid state. Said plastic state is preferably a gel-like or pasty state, said elastic solid state is preferably a gum-like state, and said non-elastic solid state is preferably a cement-like or plaster-like state.

The invention also relates to a method for the bleaching of vital or non-vital teeth by the application of the bleaching material according to the invention, wherein:

(a) when a multicomponent system is used, the components of the bleaching material are mixed;

(b) the bleaching material is applied to the teeth, into hollow spaces of the teeth, or into the cavity of the teeth; and

(c) the bleaching material solidifies at the site of application.

When a two- or multicomponent system is used, the components are mixed and subsequently applied to the teeth. The consistency of the material should be such that it can be applied, for example, with a paintbrush. Therefore, the bleaching material must have a relatively thin consistency and additionally have a sufficient total pot life.

Thereafter, the bleaching material solidifies on the teeth. The bleaching material according to the invention has the advantage that, due to its solidification, it can be removed from the teeth or, when in an elastic state, pulled off. The composition of the bleaching material is to be selected in such a way that the solidified material, although adhering well to the teeth, can be removed or pulled off from the teeth without a major expenditure of force. As a rule, the material can be integrally separated from the teeth and is discarded. The user usually does not necessarily have to remove the gel with a tooth brush, as with the removal of known materials.

Preferably, after the solidification of the bleaching material on the teeth, or at the site of application, an impression of the teeth is prepared over the bleaching material solidified on the teeth. This impression in turn is preferably used at least once for the application of additional bleaching material and thus serves the function of a bleaching bar.

The impression can be prepared with a plastic material which covers the dental surfaces on the outer (vestibular) side and on the inner (oral) side. The solidified bleaching material is included in the impression and serves as a stand-in. In particular, the use of a plastic plate or strip which can be readily conformed to the surface suggests itself. In order to ensure a sufficient pot life, the use of a photocuring material is recommendable.

The impression is detached from the teeth and removed from the mouth. The solidified bleaching material in turn can be readily detached from the teeth and from the impression and is discarded. The thus prepared impression, which has the shape of a bar, is cured with photocuring devices common in practice. Before and after the curing, the impression or bar can be processed with rotating bodies, in which case it will be available for use as a bar for the application of additional bleaching material.

The bar thus obtained is ideally suitable for the repeated application of bleaching material since it is not only fitted to the teeth, but also has cavities at the sites where the application of bleaching material is necessary, and in these cavities, larger amounts of bleaching material are present and active after the filling and inserting into the mouth.

In the bleaching method described, it is appropriate for the dentist to carry out the first application of the bleaching material, preparing a bar in the way as described. This bar can be cured directly after preparing the impression and given to the patient subsequent to the session, who will then be able to apply the bleaching material himself at home.

The method according to the invention involves a significant relieve in terms of labor and expenditure for both the dentist and the user. The home bleaching method according to the prior art requires the preparation of an impression and of a model, the preparation of the bar by a dental technician, and a second session with the dentist in which the bar is delivered and tested.

In contrast, when the bleaching material and the method according to the invention are used, a bar is made available to the user already during his first and only visit at the dentist. It is no longer required to include a dental technician. In addition, the first bleaching treatment is effected immediately together with the preparation of the bar in the mouth. Thus, the user can begin to bleach his teeth without delay, and he can possibly get an impression of the effect of the bleaching method already during his first visit at the dentist.

Further, the method according to the invention using the bleaching materials according to the invention has the advantage that, due to the structure of the bar prepared which contains indentations for bleaching material, the bleaching material is applied in a relatively specific way, using this bar, to those teeth or tooth segments which are in need of a bleaching treatment. The bleaching material does not contact, or only so in small amounts, the remainder of the oral cavity. The method according to the invention enables a selected treatment using a relatively low amount of materials and is easy on the gum, the oral cavity and the user's health.

In another embodiment of the method according to the invention, the bleaching material is applied using a bar during the first application.

In addition, the bleaching material, after solidification, can be easily and cleanly removed from the teeth and from the bar by detaching it, usually in one piece. The tedious freeing of the bar and of the teeth from tacky gel residues is not necessary.

The invention also relates to a method for the brightening of discolored non-vital teeth on the basis of the “walking bleach technique” (T. Attin, Deutsche Zahnärztliche Zeitschrift 56 (2001), pages 78ff). With root-treated teeth, discolorations of the crown frequently occur. In this case, the filling material is removed from the cavity, and a bleaching agent is applied. The bleaching agent is left within the cavity for some days, and the orifice is sealed with a provisional plastic until after a possible change of the bleaching agent.

In the corresponding method according to the invention, a dental bleaching material according to the invention which cures to a non-elastic solid state, e.g., a bleaching material comprising a phosphate, silicate or glass ionomer cement as the support material, is introduced into the cavity of the tooth to be bleached and sealed towards the oral cavity. As compared to the walking bleach technique according to the prior art, this has the advantage that the bleaching agent at the same time serves for bleaching and for sealing the cavity. This considerably facilitates the operation.

In a preferred embodiment, the bleaching material consists of a component A which is an aqueous liquid or concentrated paste with a suitable concentration of hydrogen peroxide, and a component B which is gel-like, pasty or solid and contains an alginate. After the mixing of the components A and B, the calcium ions are released from the calcium sulfate added or other substances supplying Ca²⁺ ions and lead to ionic cross-linking of the alginate polymers with the release of potassium or sodium sulfate. Surprisingly, this reaction also takes place in the presence of H₂O₂, so that a sufficient mechanical strength is achieved in acceptable curing times. In addition, several synergistic effects have a positive influence in the above described reactions:

1. The Ca ions released from Ca²⁺ supplying substances such as CaSO₄ in aqueous solutions cause, on the one hand, the above described cross-linking of the alginates and, on the other hand, the activation and release of active oxygen from hydrogen peroxide, which results in very quick build-up of a high bleaching activity which leads to short wearing times in the patient's mouth (about 30 to 240 minutes) as compared to one-component systems which have to be worn for several hours or over night.

2. The above described alginate reaction proceeds in the neutral to alkaline pH range in which the decomposition of H₂O₂ also takes place simultaneously. This means that the bleaching activity proceeds in a neutral to alkaline pH range in the patient and, as compared to bleaching systems which are active in the acidic pH range, causes a significantly lower sensitivity of the teeth/dental necks.

3. The pyrophosphates usually employed in dental alginates for reaction control act as metal chelating agents when employed in the H₂O₂ component and thus simultaneously increase the storage stability of the H₂O₂-containing component.

4. The alkali alginates employed simultaneously serve as thickeners or paste formers for a two-component paste/paste bleaching material.

5. When part of the above mentioned calcium sulfate is replaced by calcium nitrate, sodium and potassium nitrate is released by the above described reaction. These salts are known to reduce the sensitivity of the tooth.

Further, as compared to materials according to the prior art on the basis of H₂O₂ addition compounds such as carbamide peroxide, such a bleaching material has the advantage that the release of H₂O₂ is not dependent on the supply of water from the oral cavity. Therefore, the H₂O₂ can act on the teeth more uniformly and more continuously as compared to the known methods. In contrast, in the known methods, the release of H₂O₂ depends on to what extent the gel applied in the bar gets into contact with moisture.

Incidentally, a bleaching material according to the invention which contains hydrogen peroxide in a component A in aqueous solution or in aqueous gels or pastes has a storage stability which is essentially higher than that of known materials which contain H₂O₂ addition compounds as a bleaching substance. It is known that such compounds have to be stored under moistureless conditions and will lose their activity after some time or upon contact with low amounts of moisture. In contrast, aqueous hydrogen peroxide solutions of different concentrations are stable even over extended periods of time.

Since the method according to the invention is particularly specific and therefore easy on the user, and since hydrogen peroxide, for example, can be used in arbitrary concentrations and its bleaching activity does not depend on the supply of moisture from the oral mucosa, the bleaching materials according to the invention may also be used in relatively high concentrations without advantages to the user. The user is then able to achieve a successful overall treatment by comparably few individual applications. The method according to the invention also enables short application times for the individual bleaching treatments. Application over night, which is unpleasant to many users, is not necessary.

Combinations of bleaching substances are also possible. Thus, in a two-component bleaching material, an aqueous component may be provided, for example, with hydrogen peroxide. The H₂O₂ causes a high bleaching activity to be present from the beginning of the bleaching process. The second anhydrous component can contain carbamide peroxide, for example, which is released but slowly after the mixing of the components. Thus, after the initial activity of the H₂O₂ has subsided, a kind of depot effect is obtained from hydrogen peroxide released from carbamide peroxide.

In a possible variant of the method, the curing of the bleaching material is effected in the bleaching bar which is first applied to the plaster model rather than in the patient's mouth. In this way, the dentist has the possibility of individually matching, on the model, the quantity and distribution of the bleaching material to the oral situation of the patient. Only after having been fitted, e.g., by trimming with a scalpel, the cured bleaching material is inserted into the patient's mouth together with the bleaching bar.

In a further variant of the method, the bleaching material is directly applied to the teeth to be bleached. For example, after curing, a plastic or paintable photocuring plastic material is modeled or applied over the teeth covered with bleaching material. In this way, after the curing of the plastic material, an individually shaped bleaching bar is obtained already in the first bleaching process, which bar can be taken home by the patient, for example, for further home bleaching measures.

This means that the tedious preparation of the bar on indirect routes (e.g., deep-drawing process) can be dispensed with.

In the case of a simple powder/liquid system, another advantage of the bleaching materials according to the invention is their being composed of inexpensive materials common in practice. Especially when hydrogen peroxide solutions are used, a significant reduction of expenses with respect to the use of anhydrous addition compounds is achieved.

As further ionically cross-linking systems, zinc phosphate cements, carboxylate cements, silicate cements, silicophosphate cements, glass ionomer cements and glass ionomer filling materials can be employed. These materials are usually cured by admixing water and acids. Replacing the water by an aqueous hydrogen peroxide solution yields a curable cement or filler material with bleaching activity.

In another preferred embodiment, the bleaching material consists of a component A which is an aqueous liquid or paste with a suitable concentration of bleaching substance and a component B which is liquid, pasty or gel-like and contains (meth)acrylates. The curing reaction of usual chemically curing (meth)acrylate-based dental materials is based on a free-radical polymerization which is initiated by mixing the (meth)acrylate with a small amount of a free-radical initiator (e.g., benzoyl peroxide). In this case, a synergistic effect is utilized because a small portion of the bleaching substances e.g., hydrogen peroxide, which is present in a large excess as a compound having bleaching activity simultaneously adopts the role of an initiator for the free-radical polymerization of the (meth)acrylate after the mixing of the components.

Surprisingly, despite the large excess of bleaching substance, e.g., hydrogen peroxide, the polymerization reaction proceeds within realistic reaction times without the expected large exotherms.

In this application form, water-soluble (meth)acrylates or other ethylenically unsaturated compounds subject to free-radical polymerization, such as vinyl ethers, vinyl esters, N-vinyl, P-vinyl, S-vinyl, Si-vinyl compounds, allyl ethers, allyl esters or mixtures thereof, are preferably employed.

In mixtures with the above mentioned compounds, compounds of the above listed classes of substances may also be employed which are normally water-insoluble, since a mutual solubilization of water-soluble and water-insoluble compounds is possible.

Preferably employed compounds include acrylic acid, methacrylic acid, vinylsulfonic acid, acrylamidopropanesulfonic acid, vinylacetic acid, (meth)allylsulfonic acid, maleic acid, (meth)acrylamide, (meth)acrylonitrile, N-vinylpyrrolidone, vinylacetamide, alkylaminoalkyl (meth)acrylates, alkylaminopropylacrylamides, acrylamidopropyltrimethylammonium chloride, acrylamide, methacrylamide, N-vinylformamide, N-vinylacetamide, N-methyl-N-vinylacetamide, acrylonitrile, methacrylonitrile, vinyl formate, vinyl acetate, vinyl propionate, ethyl vinyl ether, butyl vinyl ether, esters of monohydric C₁- to C₁₈ alcohols and acrylic acid, methacrylic acid or maleic acid, maleic acid monomethyl ester, N-vinylcaprolactam, styrene, ethylstyrene, tert-butylstyrene, N,N′-methylene-bisacrylamide, trimethylolpropane tri(meth)acrylate, ethylene glycol di(meth)acrylate, propylene glycol di(meth)acrylate, butanediol di(meth)acrylate, hexanediol di(meth)acrylate, allyl methacrylate, polyhydric alcohols such as pentaerythritol or glycerol di- or triesterified with acrylic acid or methacrylic acid, triallylamine, tetraallylethylenediamine, divinylbenzene, diallyl phthalate, polyethylene glycol divinyl ether with polyethylene glycols having an average molecular weight of from 100 to 4000 g/mol, tri(meth)acrylates of addition products of 6 to 20 mol of ethylene oxide to 1 mol of glycerol, pentaerythritol triallyl ether, divinylurea, hydroxyethyl (meth)acrylate, hydroxypropyl (meth)acrylate, hydroxybutyl (meth)acrylate, dialkyldiallylammonium halides, allylpiperidinium bromide, N-vinylimidazole, N-vinylimidazoline, dialkylaminoalkyl (meth)acrylate, glycidyl (meth)acrylate, vinylphosphonic acid, fumaric acid, maleic anhydride, itaconic acid, 2-acrylamido-2-methylpropanephosphonic acid and its amides, hydroxyalkyl esters and esters and amides containing amino groups and ammonium groups.

Particularly preferred are acrylate and methacrylate esters of alkoxylated monohydric saturated alcohols which have been reacted with from 2 to 200 mol of ethylene oxide and/or propylene oxide per mole of alcohol, as well as monoacrylate esters and monomethacrylate esters of polyethylene glycol or polypropylene glycol, wherein the average molecular weights of the polyalkylene glycols may be up to 4000 g/mol, polyethylene glycol diacrylates and polyethylene glycol dimethacrylates, and (meth)acrylates and di(meth)acrylates of block copolymers of ethylene oxide and propylene oxide, trimethylolpropane oxyethylate tri(meth)acrylate, pentaerythritol oxyethylate tetra(meth)acrylate, glycerol ethoxylate tri (meth)acrylate.

To accelerate the free-radical polymerization, an additional polymerization initiator which decomposes into free radicals or produces free radicals under the polymerization conditions, e.g., organic peroxides and hydroperoxides, persulfates or azo compounds, can be added to component A in addition to the hydrogen peroxide present. It is preferred to use water-soluble initiators or mixtures thereof. In addition, redox initiators may be used as initiators. The oxidizing component of the redox initiator is preferably added to component A of the two-component bleaching material according to the invention. This is one of the above stated per compounds, especially hydrogen peroxide, which is contained in component A anyway.

The reducing component of the redox initiator is preferably added to component B of the two-component bleaching material according to the invention. For example, ascorbic acid, sodium ascorbate, glucose, sorbose, ammonium or alkali hydrogensulfite, alkali sulfite, alkali thiosulfate, alkali hyposulfite, alkali pyrosulfite, alkali sulfide, metal salts containing metal ions, such as copper(I), iron(II), silver(I), manganese(II), or sodium hydroxymethylsulfoxylate are used. Preferably, ascorbic acid or sodium sulfite are employed as the reducing component of the redox initiator. Mixtures of the mentioned redox initiator systems are also possible.

The component of the redox initiator having a reducing effect which is added to component B, e.g., ascorbic acid, simultaneously causes stabilization of the (meth)acrylate contained in the B component (or another echyienically unsaturated compound). During storage, the antioxidant prevents the detrimental influence of air and light and prevents premature polymerization. The ascorbic acid, for example, is completely consumed only when components A and B are mixed. This results in the formation from hydrogen peroxide of highly reactive hydroxyl free radicals which initiate the free-radical polymerization of (meth)acrylate (or another ethylenically unsaturated compound).

By suitably selecting the kinds and ratios of mono(meth)acrylates and di(meth)acrylates and/or compounds containing more than two (meth)acrylate groups, different variants of bleaching materials can be obtained. In a first variant, mixing of the liquid component A consisting of aqueous hydrogen peroxide solution in the desired concentration with the liquid component B predominantly consisting of mono(meth)acrylates, water, ascorbic acid and optionally other usual additives, such as glycerol, dyes or fragrances, from suitable mixing and dosing systems (e.g., a dual-chamber glass carpule) yields, by free-radical polymerization, a bleaching material which will solidify after a short pot life and form a gel-like consistency upon completion of the reaction. This variant has the advantage that the hydrogen peroxide component exhibits a virtually unlimited storage stability while the H₂O₂ concentration can be adjusted at any desired level.

This material is worn in a bleaching bar like a bleaching material according to the prior art. However, since it is a chemically cross-linked gel after solidification, it is significantly less water-soluble or leachable in the patient's mouth as compared to the physically thickened gels according to the prior art, and thus it is advantageous in terms of tolerance and effectiveness.

In a second variant, mixing of the liquid or pasty component A consisting of bleaching substance in the desired concentration, which may optionally be thickened by the addition of polymers, with the liquid or pasty component B consisting of a mixture of mono(meth)acrylates and di(meth)acrylates (and/or compounds containing more than two (meth)acrylate groups), water, ascorbic acid and optionally other additives, such as glycerol, silicic acids, dyes or fragrances, from suitable mixing and dosing systems (e.g., dual-chamber carpules for liquid/liquid or dual-chamber syringes ror paste/paste systems) yields, by free-radical polymerization, a bleaching material which undergoes a gradual transition from a liquid or pasty state through a gel-like state to a solid cross-linked state. During its pot life, this bleaching material can be directly applied to the dental surface. After the application, the material will cure to an elastic state and can then be covered with a plastic material, e.g., a photocuring one, in a time-saving way directly within the patient's mouth for preparing a bleaching bar. Optionally, to prevent adhesive bonding between the cured bleaching material and the photocuring plastic, a release agent can be applied.

In a third variant, which is constituted like the second variant in terms of composition and partition between the components, free-radical polymerization, upon mixing, yields a bleaching material which undergoes transition from a liquid or pasty state through a gel-like state to a solid cross-linked state and is worn in a bleaching bar by the patient. In the bleaching bar, the material cures to an elastic state and can be easily removed after the bleaching process.

In a fourth variant, which is constituted like the second variant in terms of composition and partition between the components, but wherein component B additionally contains substances for photocuring, e.g., ultraviolet initiators, such as Ciba Irgacure 184, or photoinitiators for curing with visible light, such as camphor quinone, and optionally cocatalysts, such as amino compounds, photocuring bleaching materials are obtained.

The photocuring bleaching system may also be formulated as a one-component material containing the following substances: bleaching substance, (meth)acrylates, ultraviolet initiator or photoinitiator, cocatalysts, stabilizers, and auxiliaries and additives, such as paste formers and fillers.

In the two last-mentioned embodiments, the following synergistic effect is used: the light source employed serves, on the one hand, for polymerization, and on the other hand, for activation of the bleaching substance.

One process variant also allows for curing of the bleaching material within the bleaching bar; which is first applied to a plaster model rather than within the patient's mouth. Thus, the dentist has the opportunity of adapting the quantity and distribution of the bleaching material on the model individually to the oral situation of the patient. Only after adapting the cured bleaching material, e.g., by trimming with a scalpel, it is inserted with the bleaching bar into the patient's mouth.

In a further preferred embodiment of the invention, after solidification of the bleaching material, a layer of a material for sealing the bleaching material is applied over the bleaching material, wherein said material does not contain any bleaching material and solidifies at the site of application. According to the prior art, for the coating of teeth and dental prostheses, for example in prophylaxis for the sealing of fissures, there are known coating materials adhering to the tooth (DE 37 17 762 C2, EP 0 531 483 B1) as well as dentin adhesives or dentin/enamel adhesives (DE 196 46 037 A1, DE 197 01 599, EP 0 266 548). The curable dental bleaching material according to the invention is designed in a similar way as a lacquer adhering to the tooth and contains one of the described compositions having a bleaching effect. Of the dental bleaching material applied as a lacquer and cured, the side facing the surrounding oral tissue is sealed with another lacquer having a good adhesion which does not contain any bleaching agent. Thus, it is ensured that no irritations from leaching out of the bleaching agent will occur during the wearing time of the lacquer. This embodiment is also advantageous under aesthetic aspects, because the patient wears an unobtrusive and comfortable bleaching lacquer which is adapted in color or transparent, instead of a visible bleaching bar.

Another embodiment of a chemically curing dental bleaching material according to the invention is based on the use of epimines. In the dental field, these are employed, in particular, as impression materials (DE-B-15 44 837) and as temporary crown and bridge materials (U.S. Pat. No. 34 53 242 and U.S. Pat. No. 49 03 555). In the cationic polymerization described in the above mentioned patents, polyfunctional imines (synonym of aziridine or ethyleneimine compounds) are polymerized into an elastic or rigid phase.

The epimines used in the curable dental bleaching material according to the invention can be prepared by the process described in DE-PS 15 44 837. As a cross-linking agent or catalyst for cationic polymerization, there may be used common cationizing initiators or activators, preferably arylsulfohic, acid esters according to DE-PS 15 44 837, especially 2,5-dichlorobenzenesulfonic acid methyl ester, specific sulfonium salts according to U.S. Pat. No. 4,167,618 or DE-A-2,515,593, or catalyst components containing boric acid complex according to PCT/EP 00/08568.

In an illustrative embodiment, the bleaching substance (e.g., H₂O₂ or carbamide peroxide) can be formulated together with the activator 2,5-dichlorobenzenesulfonic acid methyl ester with thickeners (e.g., Carbopol, PEG) as a liquid component A or with paste formers in a paste form. Component B preferably contains a polyalkylene glycol containing an epimine which is in a liquid or paste form, depending on the molecular weight of the polyalkylene glycol on which it is based, or on added structural builders, such as silicic acids or thickening polymers. Of course, copolymers and/or mixtures of polyethylene glycol, polypropylene glycol and polytetrahydrofuran may also be employed. After the mixing of components A and B using suitable mixing and dosing systems, a dental bleaching material is obtained which is curable by cationic polymerization.

In another embodiment of a dental bleaching material according to the invention curable by cationic polymerization, vinyl ethers are used. In DE 197 36 471 A1, PCT/EP 98/07830 and DE 197 53 461 A1, dental formulations are described which are curable by cationic polymerization, wherein the cationically polymerizable vinyl ethers which can be used are also commercially available.

Further, there is a large number of patent specifications which describe the use of vinyl ethers by cationic photocuring (e.g., EP 0 322 808, EP 608 575 A, EP 0 505 803 B1). There are used, for example, tetraethylene glycol divinyl ether, polyethylene glycol-520-methyl vinyl ether. These vinyl ethers may also be employed in combination with epoxides and/or oxetanes.

As catalysts, there are preferably used photoinitiators of the onium compound type and/or of the metallocenium compound type, respectively with a complex anion of low nucleophilicity, especially aryliodonium salts, such as diphenyliodonium tetrakis(pentafluorophenyl)borate, (3-toluyl)(dialkylphenyl)iodonium triflate, (3-toluyl) (dialkylphenyl)iodonium hexafluoroantimonate, and/or sulfonium salts, such as triarylsulfonium hexafluoroantimonate and triarylsulfonium hexafluorophosphate.

In an illustrative embodiment of the curable dental bleaching material according to the invention, the bleaching substance (e.g., carbamide peroxide) can be formulated together with the activator diphenyliodonium tetrakis(pentafluorophenyl)borate as a liquid or pasty component A (optionally thickened with thickeners, such as Carbopol or polyalkylene oxides). Component B preferably contains a polyalkylene glycol containing a vinyl ether and/or oxetane and/or epoxide which is in a liquid or paste form, depending on the molecular weight of the polyalkylene glycol on which it is based, or on added structural builders, such as silicic acids or thickening polymers. Of course, copolymers and/or mixtures of polyethylene glycol, polypropylene glycol and polytetrahydrofuran may also be employed. After the mixing of components A and B using suitable mixing and dosing systems, a dental bleaching material is obtained which is curable by cationic polymerization.

In the dental bleaching materials curing by cationic polymerization, a synergistic effect can be utilized by the use of peracids, such as phthalimidoperoxohexanoic acid. On the one hand, the acid acts as a catalyst for cationic polymerization, and on the other hand, the peracid at the same time acts as an effective bleaching substance.

In another embodiment, the curable dental bleaching material is designed as a hydrophilic polyurethane foam. From PCT/DE 92/00192, polyurethane foams are known. They consist of one or more polyether polyols, one or more diisocyanates, one or more superabsorbers, one or more accelerators for polyurethanes, water, and optionally common auxiliaries and/or additives.

The polyurethane-based curable dental bleaching material according to the invention is constituted as follows, for example:

Component A contains the bleaching agent (e.g., hydrogen peroxide solution or carbamide peroxide) and a polyether polyol. Depending on the molecular weight of the polyether polyol, component A is in a liquid or pasty form. Optionally, a thickening agent may be added to component A. Optionally, accelerators and other auxiliaries and additives are added to component A.

Component B contains a di- or polyisocyanate (e.g. hexamethylenediisocyanate, 4,4′-diisocyanatodiphenylmethane, toluylenediisocyanate (TDI), hexamethylenediisocyanate trimer). Depending on the molecular weight and viscosity of the di- or polyisocyanate, component B is in a liquid or pasty form. Optionally, a thickening agent or an accelerator for polyurethanes or other auxiliaries and additives may be added to component B.

After mixing of components A and B from suitable mixing and dosing systems (e.g., dual-chamber syringes), the dental bleaching material cures by a polyaddition reaction into a hydrophilic foam having a bleaching effect. If the above mentioned system is formulated in an anhydrous form with carbamide peroxide, a rubber-like elastomer rather than a foam will form. Of course, the above described two-component polyurethane foam system may also be formulated as a one-component polyurethane foam, comparable with the known one-component constructional foams. Instead of the monomeric di- and polyisocyanates, so-called prepolymers, e.g., isocyanate polyethers and polyesters, biurets, allophanates and cyanurates, may also be employed in order to meet toxicological criteria in the application in the dental or medical field.

In another embodiment according to the invention, the dental bleaching material is designed as a condensation-cross-linkable polysulfide (thiocol) system (Abformung in der zahnärztlichen Praxis, 3. Wirz, Gustav Fischer Verlag 1993, pages 53ff). The polysulfides are mostly pasty products wherein component A consists of a polysulfide polymer, e.g., polysulfide polyethylene glycol and optionally auxiliaries and additives, and the viscosity can be adjusted through the chain length and structure of the polymers. Component B contains the bleaching substance (e.g., hydrogen peroxide, carbamide peroxide), and optionally lead dioxide, for example, as the catalyst, sulfur as the cross-linking agent, as well as paste formers and optionally further auxiliaries and additives. Upon mixing of components A and B, cross-linking occurs from a condensation of the functional thiol groups by oxidation to release water. An elastomeric bleaching material is obtained.

In another embodiment according to the invention, a condensation-cross-linkable silicone is employed as the curing system. Component A consists of hydroxy-end-stopped polydimethylsiloxanes, the bleaching substance (e.g., carbamide peroxide or hydrogen peroxide), fillers and further auxiliaries and additives. Small amounts of water are added to component A as an initiator for the condensation reaction. Component B consists of silicate esters, a condensation catalyst (e.g., organotin compounds), and optionally further auxiliaries and additives. The viscosity can be adjusted by paste formers (e.g., silicic acid). Mixing of components A and B yields a silicone bleaching material which cures by condensation cross-linking.

Documents DE 40 10 281 A1, DE 37 41 575 A1 and EP 0 369 394 B1 describe hydrophilic addition-cross-linkable polyether impression materials which cure by platinum-catalyzed hydrosilylation. These systems can be employed for the curable bleaching material according to the invention; they have a hydrophilic polymer matrix and form a hydrophilic vulcanizate in the cured state. For example, in a two-component system, component A consists of a hydrosilylation catalyst (e.g., platinum catalyst), polyether polymers having at least two alkenyl groups in the molecule, and the substance having bleaching activity (e.g., carbamide peroxide or hydrogen peroxide). The viscosity of component A can be adjusted through the molecular weight of the polyether polymers or by the addition of paste formers or fillers. Optionally, further common auxiliaries and additives can be added. Component B consists of an SiH component (e.g., polyorganohydrogensiloxane or a siloxane-substituted polyether with SiH groups) and optionally polyether polymers with no or with at least two alkenyl groups in the molecule, and optionally further auxiliaries and additives. The viscosity of component B is adjusted through the molecular weight of the polyether polymers on which it is based, or through paste formers.

After mixing of components A and B, a curable dental bleaching material which is elastic in its final state is obtained by hydrosilylating addition cross-linking. Any gas bubbles occurring from hydrogen evolution do not adversely affect the functioning of the dental bleaching material. In this case, instead of an elastomer, an elastic foam is formed which can be removed just as comfortably. Generally speaking, the demands made on a chemically curing dental bleaching material cannot be compared with the demands made on similarly constituted basic dental materials, such as impression materials or attachment and filling materials. In this connection, it is not important that the curable bleaching material have a low shrinkage or good mechanical properties, but it must exhibit a good bleaching activity and be cured after 1 to 30 minutes and, in the case of elastomers, it must be easily removed in one piece.

### EXAMPLES

### Example 1a

Liquid Peroxide Component Of a Two-Component Bleaching Material Curable By Ionic Cross-Linking With Alginate As the Support Material

In a glass vessel, 10 parts of a 30% hydrogen peroxide solution is diluted with 40 parts of demineralized water to obtain a 6% hydrogen peroxide solution which exhibits a virtually unlimited storage stability in suitable storage packages.

### Example 1b

Powdery Alginate Component Of a Two-Component Bleaching Material Curable By Ionic Cross-Linking With Alginate As the Support Material

The alginate component is prepared by the mixing of 12 parts of sodium alginate, 9 parts of calcium sulfate dihydrate, 4 parts of magnesium oxide, 2 parts of potassium hexafluorotitanate, 0.5 parts of tetrasodium pyrophosphate, and 72.5 parts of diatomaceous earth.

### Example 1c:

Two-Component Bleaching Material Curable By Ionic Cross-Linking With Alginate As the Support Material, In the Form Of a Powder/Liquid System

Eleven parts of the powdery alginate component of Example 1b is homogeneously mixed with 50 parts of the 6% hydrogen peroxide solution of Example 1a in a plastic beaker (plaster beaker) within 30 seconds using a plastic spatula. A low-viscosity firm paste is obtained which can be readily applied to dental surfaces, e.g., with a paintbrush. After 5 minutes, the bleaching material has cured and become soft and elastic. After completion of the bleaching process, the solidified material can be removed from the teeth in one piece.

### Example 2a

Pasty Peroxide Component Of a Two-Component Bleaching Material Curable By Ionic Cross-Linking With Alginate As the Support Material

In a vacuum mixer, a thickening agent (see Table 1) is homogeneously mixed with 67 parts of 30% hydrogen peroxide solution and 13 parts of demineralized water to obtain storage-stable pastes having the viscosities and hydrogen peroxide contents as stated in Table 1.

TABLE 1

| Thickening agent | Parts | Viscosity¹⁾ at 23° C. | H₂O₂ content |
|---|---|---|---|
| Carbopol 934 P | 5 | 40,000 mPa · s | 23.6% |
| Polyvinylpyrrolidone | 50 | 30,000 mPa · s | 15.0% |
| Polyethylene glycol | 13 | 23,000 mPa · s | 21.6% |
| Na alginate | 7 | 30,000 mPa · s | 23.1% |
| Copolymer of N- | 50 | 40,000 mPa · s | 15.0% |
| vinylpyrrolidone and vinyl | | | |
| acetate | | | |
| Cross-linked vinylpyrrolidone | 20 | 40,000 mPa · s | 20.1% |

¹⁾The viscosity is measured with a Haake RS 150 viscosimeter at 23° C., oscillation frequency 1 Hz, cone-plate system, 35 mm, 4° cone, shearing stress 50 Pa; for the evaluation, the viscosity value after 80 seconds is read.

### Example 2b

Pasty Alginate Component Of a Two-Component Bleaching Material Curable By Ionic Cross-Linking With Alginate As the Support Material

In a vacuum mixer, 19 parts of potassium alginate, 18 parts of calcium sulfate dihydrate, 5 parts of magnesium oxide, 2 parts of tetrasodium pyrophosphate as well as fillers and paste formers according to Table 3 are mixed into a homogeneous paste.

TABLE 2

| | Paste | Parts of | | Parts of |
|---|---|---|---|---|
| Example | former^{a)} | paste former | Filler | filler |
| 2b-1 | PEG | 40 | — | — |
| 2b-2 | PEG | 50 | diatomaceous earth | 25 |
| 2b-3 | PPG | 50 | diatomaceous earth | 32 |
| 2b-4 | PPG | 36 | quartz | 45 |
| 2b-5 | PPG | 50 | calcium silicate | 32 |
| 2b-6 | PPG | 50 | sodium aluminum silicate | 32 |
| 2b-7 | PPG | 50 | aluminum hydroxide | 32 |
| 2b-8 | PPG | 50 | aluminum oxide | 32 |
| 2b-9 | PPG | 50 | titanium dioxide | 32 |
| 2b-10 | PPG | 50 | zinc oxide | 32 |
| 2b-11 | PPG | 50 | zeolite | 32 |
| 2b-12 | PPG | 50 | hydroxylapatite | 32 |
| 2b-13 | PPG | 50 | calcium fluorophosphate | 32 |

^{a)}PEG is polyethylene glycol having an average molecular weight of 400 g/mol. PPG is polypropylene glycol having an average molecular weight of 2000 g/mol.

### Example 2c:

Two-Component Bleaching Material Curable By Ionic Cross-Linking In a Paste/Paste Form With Alginate As the Support Material

50 parts of the pasty peroxide component of Example 2a (with sodium alginate) and 50 parts of the pasty alginate component of Example 2b-5 (with polypropylene glycol and calcium silicate) are discharged from a dual-chamber syringe through a static mixer and homogeneously mixed to obtain a low-viscosity firm paste which can be readily applied to dental surfaces, e.g., with a paintbrush. After. 5 minutes, the material has cured and become soft and elastic. After completion of the bleaching process, it can be easily removed from the teeth in one piece. This Example documents that an alginate-based paste/paste system will cure.

### Example 3a

Pasty Peroxide Component Of a Two-Component Bleaching Material Curable By Ionic Cross-Linking With Alginate As the Support Material

In a vacuum mixer, polyvinylpyrrolidone (PVP) is homogeneously mixed with 30% hydrogen peroxide solution and demineralized water (proportions see Table 3).

TABLE 3

| Example | Parts of 30% H₂O₂ | Parts of H₂O | Parts of PVP |
|---|---|---|---|
| 3a-1 | 20 | 60 | 20 |
| 3a-2 | 30 | 50 | 20 |
| 3a-3 | 40 | 40 | 20 |

### Example 3b

Pasty Alginate Component Of a Two-Component Bleaching Material Curable By Ionic Cross-Linking With Alginate As the Support Material

In a vacuum mixer, 6 parts of sodium alginate, 5 parts of highly dispersed hydrophobized silicic acid having a BET surface of 140 m²/g, 5 parts of calcium sulfate dihydrate, 0.25 parts of tetrasodium pyrophosphate, 50-parts of polypropylene glycol having an average molecular weight of 2000 g/mol, and a powdery bleaching agent and a filler according to Table 4 are homogeneously mixed.

TABLE 4

| Example | Bleaching agent | Parts | Filler | Parts |
|---|---|---|---|---|
| 3b-1 | carbamide peroxide | 28 | calcium silicate | 11 |
| 3b-2 | sodium percarbonate | 30 | calcium silicate | 9 |
| 3b-3 | sodium perborate | 30 | calcium silicate | 9 |
| 3b-4 | potassium peroxodisulfate | 10 | calcium silicate | 29 |
| 3b-5 | phthalimidoperoxohexanoic | 10 | calcium silicate | 29 |
| | acid | | | |
| 3b-6 | none | 0 | calcium silicate | 39 |

### Example 3c:

Two-Component Bleaching Material Curable By Ionic Cross-Linking In a Paste/Paste Form With Alginate As the Support Material

80 parts of the pasty peroxide component of Example 3a-2 and 20 parts of the pasty alginate component of Example 3b-1 are discharged from a dual-chamber syringe through a static mixer and homogeneously mixed to obtain a low-viscosity firm paste which can be readily applied to dental surfaces, e.g., with a paintbrush. After 5 minutes, the material has cured and become soft and elastic. After completion of the bleaching process, it can be easily removed from the teeth in one piece.

The compressive strength of the cured bleaching material is 0.43 MPa according to ISO 1563, and its recovery from deformation is 97.0% according to ISO 1563. These values meet the minimum requirements of ISO 1563 for dental alginate impression compositions of 0.35 MPa for the compressive strength and 95.0% for the recovery from deformation.

The bleaching agent has a high initial bleaching activity due to the hydrogen peroxide, and a high long-term bleaching activity due to the carbamide peroxide. Thus, it is suitable for the so-called “jump start” bleaching in which the series of home bleaching treatments is opened in the dental practice by an intensive treatment.

In a way analogous to that of the executed Example 3c, further combinations of peroxide pastes and alginate pastes from Tables 3 and 4 can be mixed and employed as a curable dental bleaching material. In this way, different bleaching intensities and indication fields can be reached with the curable dental bleaching material according to the invention.

The mixing ratio between the two components may be varied between 1:1 and 1:10, mixing ratios of 1:1, 1:2, 1:4 and 1:5 being particularly preferred.

### Example 4a

Liquid Peroxide Component Of a Two-Component Bleaching Material Curable By Free-Radical Polymerization With Poly(Meth)Acrylates As the Support Material

The peroxide component is prepared by diluting 23 parts of a 30% hydrogen peroxide solution with 77 parts of demineralized water to obtain a 7% hydrogen peroxide solution which exhibits a virtually unlimited storage stability in suitable storage packages.

### Example 4b

Liquid (Meth)Acrylate Component Of a Two-Component Bleaching Material Curable By Free-Radical Polymerization With Poly(Meth)Acrylates As the Support Material

In a light-protected vacuum protective gas apparatus, 72 parts of polyethylene glycol monomethyl ether methacrylate having an average molecular weight of 475 g/mol, 8 parts of polyethylene glycol dimethacrylate having an average molecular weight of 400 g/mol, 2 parts of ascorbic acid and 18 parts of water are mixed into a homogeneous solution under argon as a protective gas. The solution prepared is stored in the absence of light.

### Example 4c:

Two-Component Bleaching Material Curable By Free-Radical Polymerization With Poly(Meth)Acrylates As the Support Material, In the Form Of a Liquid/Liquid System

50 parts of the liquid peroxide component of Example 3a and 50 parts of the liquid methacrylate component of Example 3b are homogeneously mixed, for example, in a glass carpule, and discharged to obtain a bleaching material which undergoes a gradual transition from a liquid through a gel-like to a solid elastic state from free-radical polymerization. During the pot life, the bleaching material can be directly applied to the dental surfaces, or introduced into a bleaching bar. After the bleaching process is complete, the solidified material can be removed from the teeth or out of the bleaching bar in one piece.

### Example 5a

Pasty Peroxide Component Of a Two-Component Bleaching Material Curable By Free-Radical Polymerization With Poly(Meth)Acrylates As the Support Material

In a vacuum mixer, 67 parts of 30% hydrogen peroxide solution is homogeneously mixed with 3 parts of water and 30 parts of a polyviriylpyrrolidone having a molecular weight of 1,100,000 g/mol to obtain a colorless transparent paste having a viscosity of 60,000 mPa·s.

### Example 5b

Pasty (Meth)Acrylate Component Of a Two-Component Bleaching Material Curable By Free-Radical Polymerization With Poly(Meth)Acrylates As the Support Material

In a light-protected vacuum mixer, 55 parts of polyethylene glycol monomethyl ether methacrylate having an average molecular weight of 475 g/mol, 5 parts of polyethylene glycol dimethacrylate having an average molecular weight of 400 g/mol, and 20 parts of hydrophilic highly dispersed silicic acid having a BET surface of 200 m²/g are homogeneously mixed with a solution of 2 parts of ascorbic acid in 18 parts of demineralized water to obtain a slightly opaque paste which must be filled and stored in the absence of light and air.

### Example 5c:

Two-Component Bleaching Material Curable By Free-Radical Polymerization With Poly(Meth)Acrylates As the Support Material, In the Form Of a Paste/Paste System

50 parts of the pasty peroxide component of Example 4a and 50 parts of the pasty methacrylate component of Example 4b are discharged from a dual-chamber syringe through a static mixer and homogeneously mixed to obtain a bleaching material which undergoes a gradual transition from a pasty through a gel-like to a solid elastic state from free-radical polymerization. During the pot life, the bleaching material can be directly applied to the dental surfaces. After the bleaching process is complete, the solidified material can be removed from the teeth or out of the bleaching bar in one piece.

### Example 6

Example Of a Curable Bleaching Material With a Silicophosphate Cement As the Support Material

45 parts of a commercially available silicophosphate cement (Zhanelka zinc porcelain cement supplied by Speiko) is homogeneously mixed with 20 parts of a 10% hydrogen peroxide solution to obtain an opaque cement which can be used as a bleaching filling in molars or premolars, for example, for the internal bleaching of root-canal-treated discolored teeth.

### Example 7

Example Of a Curable Bleaching Material With a Polycarboxylate Cement As the Support Material

50 parts of a commercially available polycarboxylate cement (Poly-F-Plus supplied by De Trey) is homogeneously mixed with 10 parts of a 10% hydrogen peroxide solution using a spatula on a mixing block to obtain a cement with bleaching activity for attachment, for rebasing, for basic filling or for provisional filling, for example, for the internal bleaching of root-canal-treated discolored teeth.

### Example 8

Example Of a Curable Bleaching Material With a Glass Ionomer Cement As the Support Material

30 parts of a commercially available glass ionomer cement (Aquacem supplied by De Trey) is mixed with 10 parts of a 10% hydrogen peroxide solution using a spatula on a mixing block to obtain an attachment cement with bleaching activity, for example, for the internal bleaching of root-canal-treated discolored teeth.

### Example 9

Example Of a Curable Bleaching Material With a Glass Ionomer As the Support Material

50 parts of a commercially available glass ionomer filling material (Ketac Fil Plus supplied by Espe) is mixed with 50 parts of a 10% hydrogen peroxide solution using a spatula on a mixing block to obtain a tooth filling material with bleaching activity, for example, for the internal bleaching of root-canal-treated discolored teeth.

### Example 10

Example Of the Bleaching Method According To the Invention

The bleaching material according to Example 1c is applied to the teeth to be bleached in one continuous bead using a paintbrush. After five minutes from the application, the material has cured to a soft elastic state and sticks to the dental surface. Subsequently, the dental surface of the whole jaw is covered by a plastic photocuring material, e.g., Convertray transparent of Wilde Dental GmbH, on the outer (vestibular) side, on the masticatory surface and on the inner (oral) surface, and the material is adapted to the surface. The applied cured bleaching material simultaneously serves as a stand-in. With a commercially available light curing device (type Dent-Lite), the thus shaped bar is cured.

After removal from the mouth, the bar is processed with rotating bodies and given to the patient. The cured bleaching material can usually be removed from the dental surface in one piece or remains adhered in the bar from which it can also be easily removed. From the dentist, the patient receives a sufficient amount of bleaching material; he compounds it at home and applies it within the bar. Usually, a patient wears the bar once to twice a day for 30 minutes each. Now, after each change, he can easily remove the cured bleaching material.

### Example 11

Example Of the Bleaching Method According To the Invention

For preparing a bleaching bar, an impression of the upper and lower jaws is prepared by the dentist, for example, using a commercially available alginate impression material (e.g., Xanthalgin select supplied by Heraeus-Kulzer). In the laboratory, a plaster model is prepared from this impression. Subsequently, a stand-in material is applied to the plaster in the region of the teeth to be brightened. Thereafter, a bar is prepared by a deep-drawing method from plastic plates on the thus prepared plaster model and adapted. The dentist fills this bar with one of the curable bleaching materials according to any of Examples 1c, 2c, 3c or 4c. The bar filled with curable bleaching material is first applied to the plaster model. On the plaster model, the quantity and distribution of the bleaching material can be controlled very accurately. After curing, overflowing regions can be trimmed. The thus processed bleaching material is applied to the patient's mouth within the bleaching bar. In this way, the bleaching material can be applied to the desired places very selectively and safely. Thus, an effective and patient-friendly bleaching is achieved which is easy on the tissue.

### Comparative Example 1

In a vacuum plaster mixer, 30 g of a commercially available dental superhard plaster Fuji Rock of GC is homogeneously mixed with 6 ml of 30% hydrogen peroxide solution. The plaster has a similar consistency as if compounded with 6 ml of water, but does not cure. After a few hours, any water present slowly evaporates, and a strong, but very brittle mass remains which does not compare to plaster cured with water.

This Example shows that it is not a matter of course that the water can be replaced by hydrogen peroxide or hydrogen peroxide solutions in substances curable by the addition of water. Rather, it is surprising that the curing reactions of Examples 1c, 2c, 3c and 4c according to the invention are not adversely affected in a critical way by the partial use of hydrogen peroxide instead of water.

1. A dental bleaching material containing at least one bleaching substance and at least one support material capable of solidifying by a chemical reaction.

2. The bleaching material according to claim 1, characterized by containing hydrogen peroxide, carbamide peroxide, phthalimidoperoxohexanoic acid, alkali percarbonate, alkali perborate, alkali peroxodisulfate, peroxyacetic acid, alkali hypochlorite, addition compounds of hydrogen peroxide and/or peroxides of organic acids as said bleaching substance.

3. The bleaching material according to claim 1 and/or 2, characterized by containing alginates, alginic acids, acrylates, methacrylates, acrylic acid, methacrylic acid, acrylamide, vinyl acetate, N-vinylpyrrolidones, methyl vinyl ether maleate, aziridines, vinyl ether, epoxides, polyols, polyamines, di- and polyisocyanates, cyanoacrylates, hydroxypolydialkylsiloxanes, alkenyl-substituted polyethers and polysiloxanes, polyethers containing SiH and Si-vinyl, polysulfides, glass ionomer filling materials, glass ionomer cements, zinc phosphate cements, carboxylate cements, silicate cements and/or silicophosphate cements as said support material.

4. The bleaching material according to at least one of claims 1 to 3, characterized by additionally containing reinforcing and/or non-reinforcing fillers, reaction inhibitors, di- or polyvalent salts, substances supplying Ca²⁺, calcium sulfate, wetting agents, surfactants, emulsifiers, alcohols, water, solvents, buffer systems, fragrances, flavors, gelling agents, substances for increasing the viscosity (thickeners and paste formers), glycols, glycerol, polyethylene glycols, propylene glycols, diethylene glycols, polypropylene glycols, polyurethanes, fatty alcohol ethoxylates, polyvinylpyrrolidone, copolymers of N-vinylpyrrolidone and vinyl acetate, polyacrylic acid or its alkali or alkaline earth salts, carboxymethyl-, methyl-, hydroxyethyl-, hydroxypropylcellulose, polysaccharides, silica, kieselguhr, diatomaceous earth, substances for photocuring, ultraviolet initiators and photoinitiators, cross-linking catalysts, colorants, color pigments and/or colorant systems which undergo a color change upon changes of pH or redox potential.

5. The bleaching material according to at least one of claims 1 to 4, characterized by consisting of at least two components A and B.

6. The bleaching material according to claim 4, characterized in that component A contains said at least one bleaching substance and component B contains said at least one support material.

7. The bleaching material according to at least one of claims 1 to 6, characterized in that said bleaching material and/or said components are in the form of a gel, liquid, powder or paste.

8. The bleaching material according to claim 6, characterized in that components B and A are in the forms, respectively, of a gel and a gel, a liquid and a liquid, a powder and a powder, a paste and a paste, a gel and a liquid, a gel and a paste, a paste and a liquid, or a powder and a paste.

9. The bleaching material according to at least one of claims 1 to 8, characterized by being in a plastic, non-elastic solid or elastic solid state after solidification of said support material.

10. A method for the bleaching of vital or non-vital teeth by applying a bleaching material according to at least one of claims 1 to 9, characterized in that:
(a) when a multicomponent system is used, the components of the bleaching material are mixed;
(b) the bleaching material is applied to the teeth, into hollow spaces of the teeth, or into the cavity of the teeth; and
(c) the bleaching material solidifies at the site of application.

11. The method according to claim 10, comprising, after step (c), a step
(d) in which an impression of the teeth having the function of a bleaching bar is prepared together with the bleaching material solidified on the teeth at the site of application.

12. The method according to claim 11, comprising a step
(e) in which the prepared impression of the teeth having the function of a bleaching bar is used for the application of bleaching material at least once.

13. The method according to claim 10, characterized in that the bleaching material is applied by means of a bar in step (b).

14. The method according to claim 10, characterized in that a layer of a material for sealing the bleaching material is applied over the solidified bleaching material, wherein said material does not contain any bleaching material and solidifies at the site of application.
